(19) United States
(12) Patent Application Publication
(10) Pub. No.: US 2009/0112195 A1
(43) Pub. Date: Apr. 30, 2009

(54) Means for Calculating an Operating Parameter (Energy, Power or Duration of Emission) of an Endovenous Laser

(75) Inventor: Jaouad Zemmouri, Genech (FR)

Correspondence Address: HARNESS, DICKEY & PIERCE, P.L.C., P.O. BOX 828, BLOOMFIELD HILLS, MI, 48303, US

(73) Assignee: OPTICAL SYSTEM & RESEARCH FOR INDUSTRY AND SCIENCE OSYRIS, Hellemes (FR)

(21) Appl. No.: 11/922,642

(22) PCT Filed: Jun. 22, 2006
(86) PCT No.: PCT/FR2006/001417

§ 371 Date: Apr. 3, 2008

(30) Foreign Application Priority Data
Jun. 23, 2005 (EP) 05370015.9

(51) Int. Cl. A61B 18/20

(52) U.S. Cl. 606/12

(57) ABSTRACT

The invention concerns a means for calculating an operating parameter of an endovenous laser delivering a value of the energy (E) of the laser beam, or a value of the power (Pₗₐₛₑᵣ) of the laser beam for a predetermined duration of emission (tₗₐₛₑᵣ), or a value of the duration of emission (tₗₐₛₑᵣ) of the laser beam for a predetermined power (Pₗₐₛₑᵣ) of the laser beam, said value being based on an exponential law which depends on the interior diameter (or radius) of the vein to be treated.

### TECHNICAL FIELD

The present invention concerns the field of endovenous lasers and their use for the local treatment of blood-containing veins and in particular for locally collapsing or plugging of veins. It finds its principal, but not its only, application in the field of treatment of varicose veins

### PRIOR ART

For the treatment of veins and in particular, of varicose veins by their local collapsing or plugging, the use of endovenous lasers, which are designed for introduction into the interior of a vein is known at present. In practice, these lasers are introduced into the vein for example by the insertion of a catheter and an optical fiber as far as the part of the zone to be treated and when the laser has been positioned in the interior of the vein, the shots are fired either continuously or discontinuously, gradually withdrawing the laser over a distance which is a function of the length of the zone to be treated. The wavelengths usually employed lie within the 800 to 1000 nm band.

The use of an endovenous laser is preferable to the application of non-invasive surface laser treatment, because it makes possible more effective treatment coupled with a lower risk of collateral damage of other tissues and makes it possible to destroy the targeted veins to a greater depth.

In the American patents U.S. Pat. No. 4,564,011 and U.S. Pat. No. 5,531,739, the endovenous laser is used for the local coagulation of blood inside a vein and for the complete or partial local plugging of the vein. It is because of the basic approach of these methods that the vein is not evacuated of its blood content.

U.S. Pat. No. 6,398,777 sets out the use of an endovenous laser for the local destruction of endothelial cells and of the internal wall of the vein in such a way as to achieve a local fibrosis of the vein, which makes it possible to reduce the internal diameter of the said vein and if necessary, to collapse it entirely.

According to the teaching of U.S. Pat. No. 6,398,777, in order to achieve the desired effect of fibrosis of the vein, it is essential prior to laser treatment to evacuate the blood present in the vein to be treated and to bring the laser into contact with the interior wall of the vein in question. On the one hand, this method involves a supplementary operation, namely, that of evacuating the vein and the operation of positioning the laser in contact with the interior wall of the vein, which is a very delicate operation. On the other hand and most importantly, it is found in practice that this method involving the evacuation of the vein, cannot be used because of the major risk of irreversible destruction of healthy skin tissues adjacent to the zone of the vein to be treated. Thus, in contrast to what is recommended in U.S. Pat. No. 6,398,777, it is found in the endovenous laser treatment of a vein, that the treated vein still contains blood.

Whatever is the method used when employing an endovenous laser, a practitioner's major difficulty resides in selecting the energy of the laser, which it is necessary to use. Excessively low energy leads to ineffective treatment, whilst excessively high energy may lead to the irreversible destruction of healthy tissues adjacent to the vein.

Currently, the setting of the energy of a laser beam, namely of the power and/or the time of the laser emission, is performed empirically by the practitioner on the basis of his experience and there is accordingly an unsatisfied need for being able to determine easily and safely the energy of an endovenous laser for the local treatment of a blood-containing vein with a view to wholly or partially plugging the said vein.

### OBJECT OF THE INVENTION

It is one of the objectives of the invention to provide a simple and effective solution for facilitating the setting of an endovenous laser used for the treatment of a blood-containing vein with the view of wholly or partially collapsing or plugging the said vein.

### SUMMARY OF THE INVENTION

The inventors have shown that surprisingly there is a simple relationship between the laser energy required for endovenous treatment of a blood-containing vein and the cross-section, in particular of the diameter or radius of the said vein prior to treatment.

On the basis of this finding, the first objective of the invention is the provision of a means of calculation of a parameter of functioning of an endovenous laser.

Characteristically, the means of calculation furnishes a value of energy (E) of the laser beam, or a value of the power (Pₗₐₛₑᵣ) of the laser beam for a predetermined time of emission (tₗₐₛₑᵣ), or the value of the time of emission (tₗₐₛₑᵣ) of the laser beam for a predetermined power (Pₗₐₛₑᵣ) of the laser beam, the said value being a function of an exponential law, which depends on the internal diameter or radius of the vein to be treated.

Preferably, the means of calculation shall be designed for determining the energy of the laser beam using the following equation: E=k1e^{k2.Dvein}, or for determining the power (Pₗₐₛₑᵣ) of the laser beam using the following equation: Pₗₐₛₑᵣ=(k1/tₗₐₛₑᵣ)e^{k2.Dvein}, or for determining the time of emission (tₗₐₛₑᵣ) of the laser beam using the following equation: tₗₐₛₑᵣ=(k1/Pₗₐₛₑᵣ)e^{k2.Dvein}, with Dᵥₑᵢₙ representing the internal diameter of the vein, k1 and k2 being predetermined constants.

The values of k1 and k2 depend in particular on the laser wavelength. Preferably, k1 lies between 5 and 8 and k2 between 0.4 and 0.6.

In a variant of the embodiment, the means of calculation is implemented in the form of a computer programme recorded on a memory support, or in an electronic memory.

In another variant of the embodiment, the means of calculation is an abacus.

The invention likewise has the further object of providing an endovenous laser comprising a means of automatic calculation of the value of energy (E) of the laser beam, or of the value of the power (Pₗₐₛₑᵣ) of the laser beam for a predetermined time of emission, or of the value of the time of emission (tₗₐₛₑᵣ) of the laser beam for a predetermined power of the laser beam, the said means of calculation having the aforementioned characteristics.

The invention has a yet further object of a process of the setting of an endovenous laser, according to which, the value of energy (E) of the laser beam, or the value of the power (Pₗₐₛₑᵣ) of the laser beam for a predetermined time of emission, or the value of the time of emission (tₗₐₛₑᵣ) of the laser beam for a predetermined power of the laser beam, is setting on the basis of a measurement of the cross-section of the vein to be treated and by using a means of calculation according to the invention.

The invention likewise has the object of providing a process of treatment of a vein by means of a laser beam, which is introduced into the interior of the blood-containing vein as far as the site to be treated.

According to this process, the cross-section of the vein is measured at the site to be treated and the laser is set according to the aforementioned process of setting.

### BRIEF DESCRIPTION OF THE FIGURES

Other characteristics and advantages of the invention will emerge more clearly from reading the detailed description which follows and which is given as a non-limiting and non-exhaustive example of the invention and from a reference to the appended drawings, where:

FIG. 1 is an example of an abacus according to the invention furnishing the laser energy as a function of the internal diameter (Dᵥₑᵢₙ) of the vein,

FIG. 2 is a schematic representation of a vein and the laser fiber introduced into the vein,

FIG. 3 is a cross-section of the vein in FIG. 2,

FIGS. 4 to 10 are iso-damage curves obtained in respect of different internal vein radii,

and FIG. 11 is a synoptic of a laser equipped with means of automatic setting specific to the invention

### DETAILED DESCRIPTION

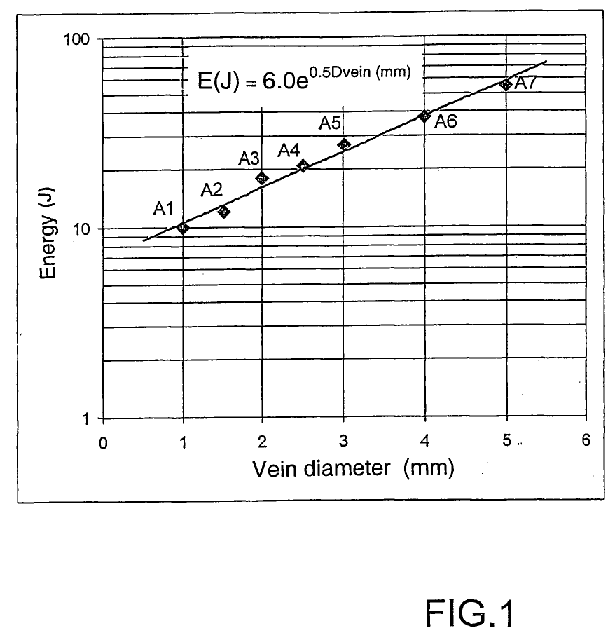

FIG. 1 is an abacus which, in the field of endovenous laser treatment of veins, provides an example of a simple relationship between the energy E required for the laser beam and the cross-section of the vein to be treated and more specifically in this example, the internal diameter Dᵥₑᵢₙ of the vein to be treated. This relationship in FIG. 1 was obtained by a digital calculation based on the geometrical model below and by means of the method of digital calculation set out below.

### Geometrical Model

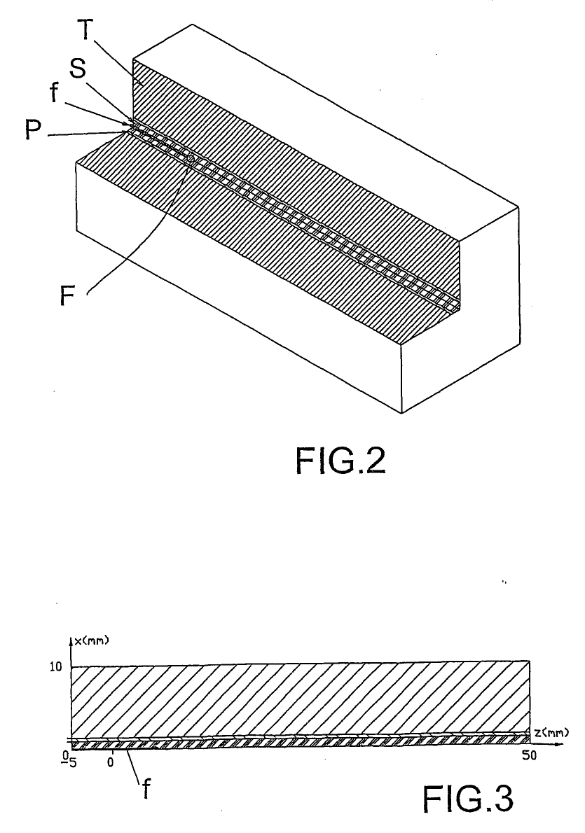

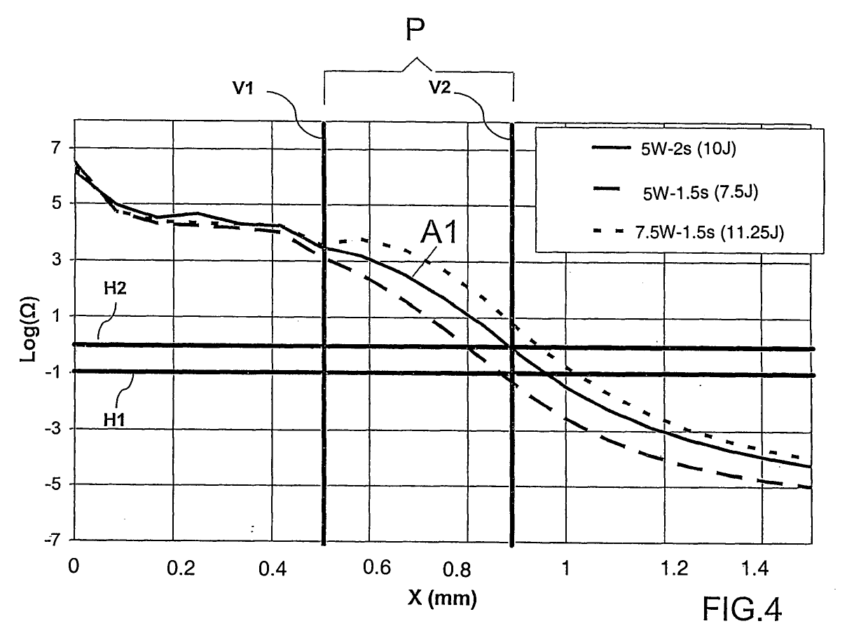

The geometrical model in FIGS. 2 and 3 is used for these calculations. In this model, the vein to be treated is a cylinder comprising a wall (P) containing blood (S) and which is surrounded by tissue (T). In FIG. 2, the laser fiber is shown schematically and carries the reference (f).

Given that the system has a revolution symmetry centred on the longitudinal axis of the vein, the calculations are carried out in a two-dimensional section with FIG. 3 parameters (the thickness of the wall of the vein being Eᵥₑᵢₙ and the internal radius of the vein being Rᵥₑᵢₙ).

In FIG. 2, the laser optical fiber is shown schematically and carries the reference (f), with the laser beam F from the said optical fiber f being centred on the central longitudinal axis of the vein and the wavelength of the laser beam F lying between 800 and 1000 nm and has, for example, a value of 980 nm.

For calculating the distribution of the laser beam in space, it is assumed that the propagation of the laser beam is mainly implemented by the medium (dominant diffusion action), as taught by the publication of M. N. Lizuca, I. A. Vitkin, M. C. Kolios and M. D. Sherar entitled “The effects of dynamic optical properties during interstitial laser photocoagulation”, Phys. Med. Biol. 45 (2000)1335-1357.

The distribution of the power emitted at a given point is given by the following equation:

$$φ ( r ) = \frac{{P}_{Laser} \cdot exp ( - {μ}_{eff} \cdot r )}{4 π \cdot D \cdot r} \qquad ( 1 )$$

### Where:

Pₗₐₛₑᵣ is the power of the laser beam (Watts);

μ_{eff} is the effective absorption coefficient (mm⁻¹);

r is the distance (mm) from the point of emission of the laser beam (i.e. in practice the point of output of the laser fiber);

D characterises the diffusion (mm).

μ_{eff} is defined by the following equation:

μ_{eff}=\sqrt{3·μₐ(μₐ+μ′ₛ)} (2)

### Where:

μₐ is the coefficient of absorption (mm⁻¹);
μ′ₛ is the reduced coefficient of diffusion which is defined by the following equation:

μ′ₛ=μₛ·(1−g), (3)

### Where:

μₛ is the coefficient of diffusion (mm⁻¹);
g is the factor of anisotropic diffusion (no units).

The values of μₐ and μ′ₛ vary with the wavelength of the laser beam.

D is defined by the following equation:

$$D = \frac{1}{3 ( {μ}_{a} + {μ}_{s}^{′} )} = \frac{{μ}_{a}}{{μ}_{eff}^{2}} \qquad ( 5 )$$

and r is defined by the following formula:

r=\sqrt{x²=z²} (6)

### Where:

x (mm) is the radial distance measured along the axis X (FIG. 3) from the point of emission of the laser beam;
z (mm) is the longitudinal distance measured along the axis Z (FIG. 3) from the point of emission of the laser beam.

The power absorbed (W/mm³) at each point is calculated using the following formula:

P_{abs}=μₐ·φ(r) (7)

It is assumed for the purpose of the calculation that the first laser shot is always fired at the point of the coordinates (0,0) of FIG. 3. When the “multi-pulse” mode is simulated, a variable z′ is introduced, which represents the relative position in the vein and which is defined by the following formula:

z′=z−z_{inc} (8)

### Where:

Z_{inc} (mm) represents the absolute position.

To calculate the absolute position Z_{inc} of each shot along the longitudinal axis Z, a counter is used, which is multiplied by the distance between each shot and the next, being distance of withdrawal of the fiber (f) of the laser.

In the case of a shot called continuous with a simultaneous and gradual withdrawal of the fiber (f) of the laser, the variable Z_{inc} in the foregoing formula (8) is replaced by the formula:

Z_{inc}=t·v, (9)

### Where:

v is the speed of withdrawal of the fiber and t is the time of withdrawal.

### Optical Parameters

Only the energy of the laser [laser power (Pₗₐₛₑᵣ) and/or the time of emission of the laser] is a variable; the values of the other parameters being fixed according to table 1 below and are defined for a wavelength of 980 nm:

TABLE 1

| Medium | μₐ(mm⁻¹) | μₛ (mm⁻¹) | μ_{eff} (mm⁻¹) |
|---|---|---|---|
| Blood (S) | 0.25 | 0.60 | 0.80 |
| Wall (P) | 0.1 | 2.0 | 0.80 |
| Tissue (T) | 0.030 | 1.0 | 0.30 |

### Thermal Parameters

For the calculation of the temperature rise and of thermal diffusion, the heat equation below is used:

$$∇ \cdot ( k \cdot ∇ T ( r , t ) ) + {P}_{abs} = {C}_{p} \cdot \frac{\partial T ( r , t )}{\partial t} \qquad ( 10 )$$

### Where:

T(r,t) is the temperature at a given point and at a given moment;
k (W·mm⁻¹·K⁻¹) is the thermal conductivity;
Cₚ (J·mm⁻³·K⁻¹) is the thermal volume capacity defined by the following formula:

Cₚ=C·ρ (11)

### Where:

C (J·g⁻¹·K⁻¹) is the mass thermal capacity and,
ρ(g·mm⁻³) is the density of the medium.

These parameters are set by the values of table II below, the ambient temperature being set at 37° C.:

TABLE II

| Medium | C (J · g⁻¹ · K⁻¹) | ρ (g · mm⁻³) | k (W · mm⁻¹ · K⁻¹) |
|---|---|---|---|
| Blood (S) | 3.82 | 1.05 · 10⁻³ | 5.6 · 10⁻⁴ |
| Wall (P) | 3.78 | 1.05 · 10⁻³ | 5.6 · 10⁻⁴ |
| Tissue (T) | 3.78 | 1.05 · 10⁻³ | 5.6 · 10⁻⁴ |

### Transition of the Blood Phase

Given that like most other tissues, blood consists of a high percentage of water, above 100° C. blood undergoes a phase transition. Given that the vein is a closed, but deformable medium, it is difficult for blood temperature to exceed 100° C. Therefore, a limitation has been introduced into the model consisting in markedly increasing the thermal capacity when the temperature of the medium exceeds 100° C.

### Calculation of Iso-Damage Curves

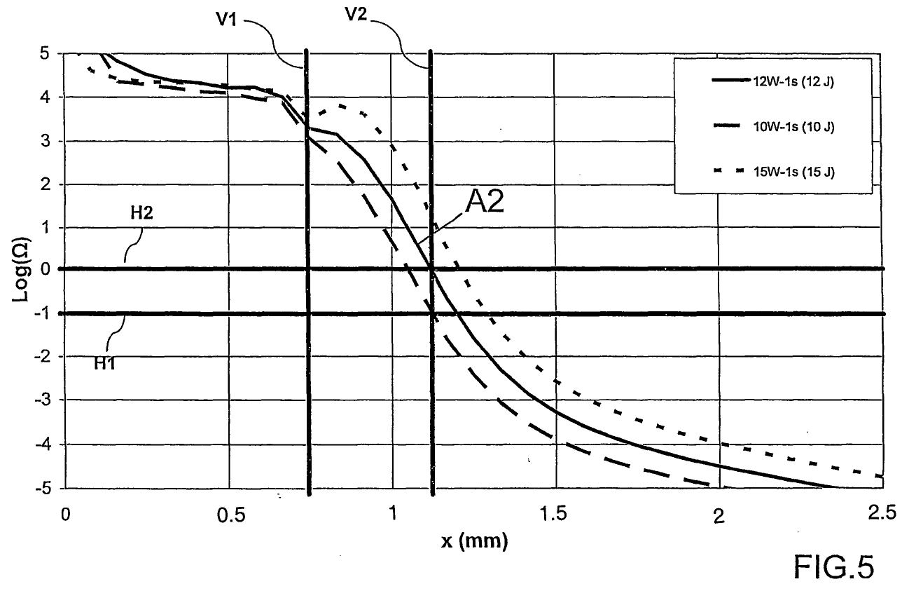

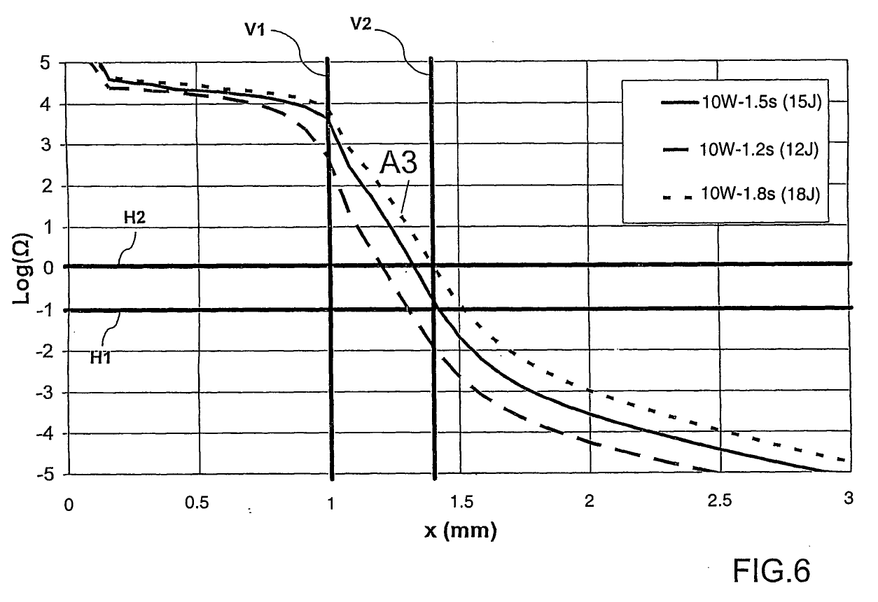

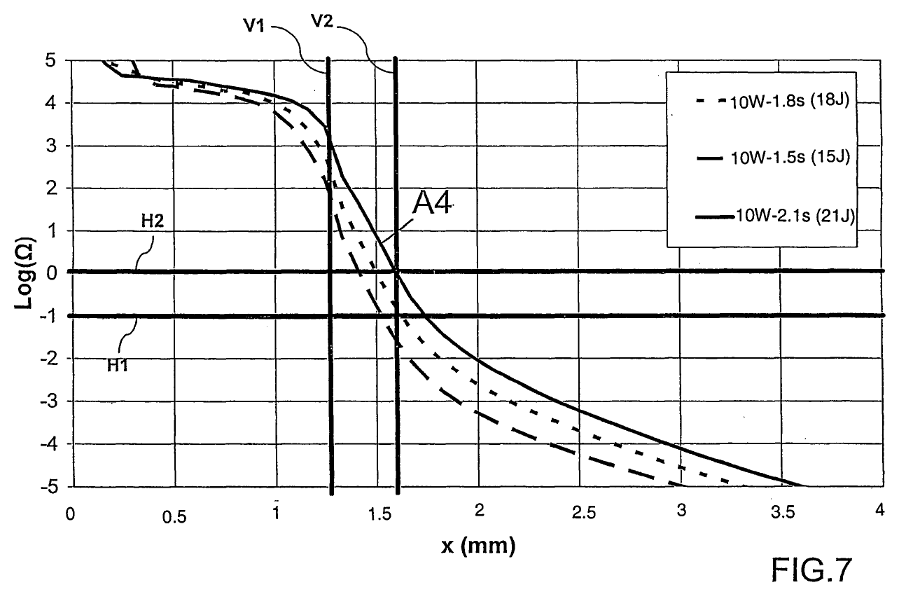

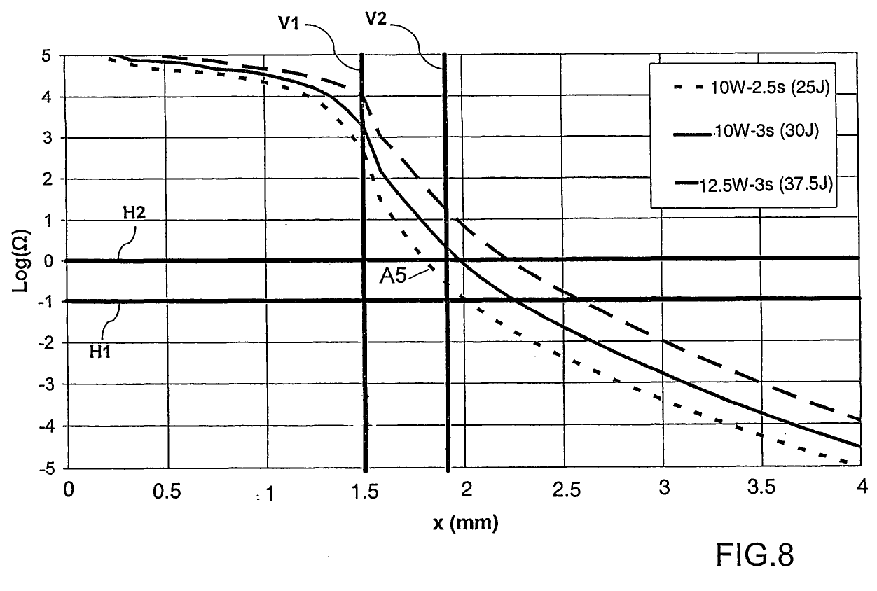

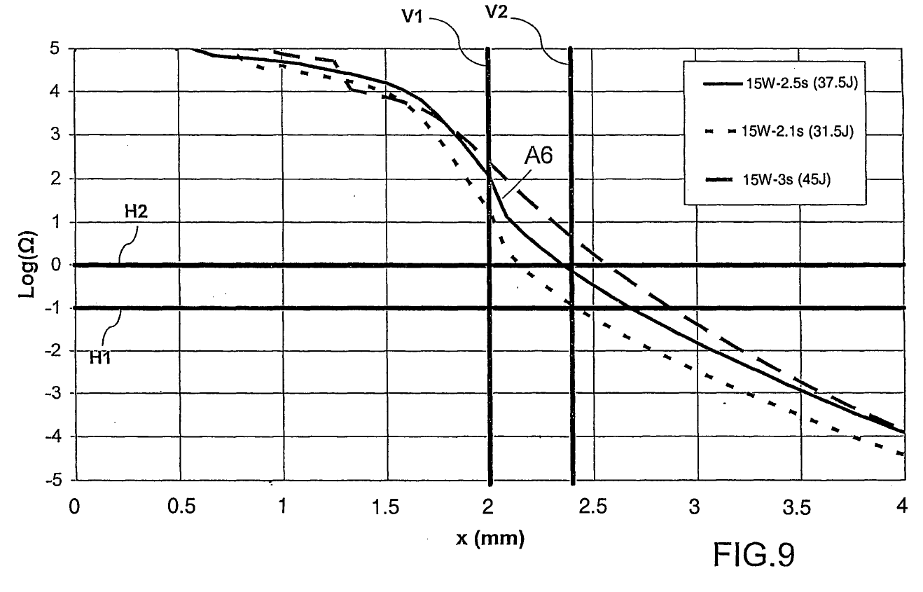

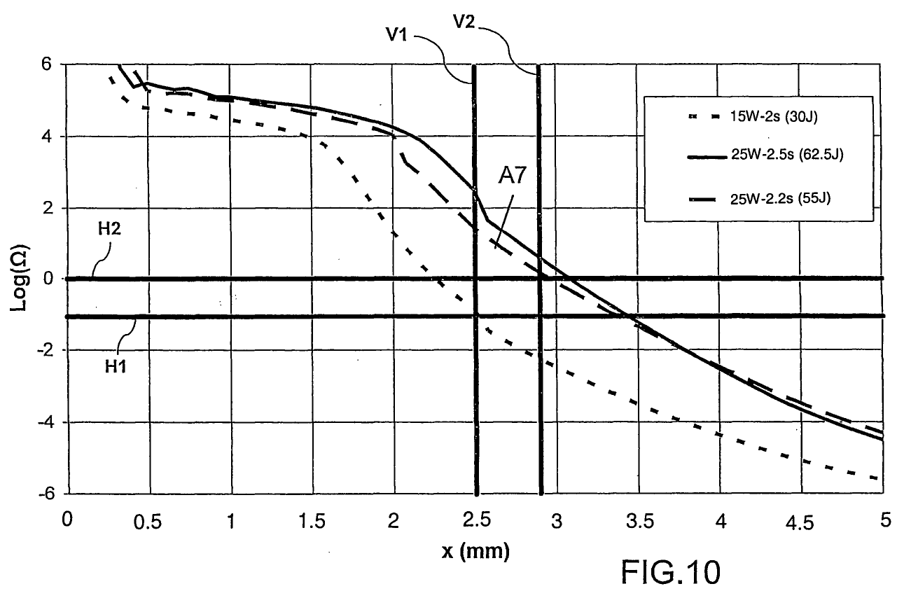

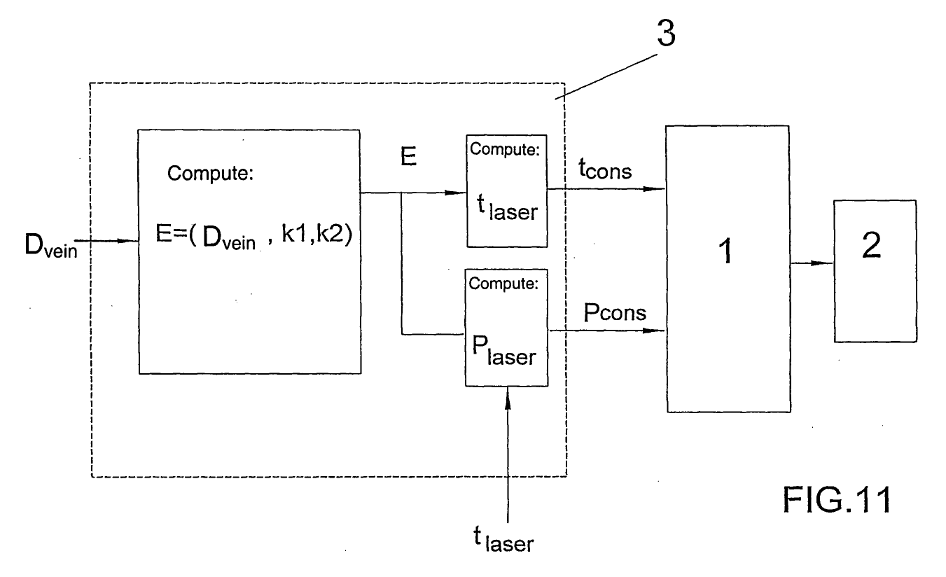

Iso-damage curves are determined by digital calculation in FIGS. 4 to 10 for different internal radii of a vein. FIGS. 4 to 10 are iso-damage curves for veins of respective internal radii of 0.5, 0.75, 1, 1.25, 1.5, 2 and 2.5 mm. Each iso-damage curve in FIGS. 4 to 10 is calculated for a specific pair [laser beam power (Watts)−time of emission of the laser beam (seconds)].

In order to calculate the said iso-damage curves in FIGS. 4 to 10, the Arrhenius formula below is used, according to which the effect of temperature on a tissue depends solely on two parameters, namely, on the value of the temperature and on that of the time during which that temperature is maintained.

$$log ( Ω ) = log ( A ) + log [ {\int }_{0}^{t} exp ( \frac{- Ea}{R \cdot T ( r , t )} ) d t ] \qquad ( 12 )$$

The parameter Ω can also be expressed by the following formula:

$$Ω = - ln \frac{C ( t )}{{C}_{0}} \qquad ( 13 )$$

### Where:

Cₒ is the initial concentration of tissue cells,
C(t) is the concentration of undamaged cells at moment t.

In respect of the criterion normally used for determining the zone of maximum damage, the fact should be taken into account that when two-thirds (˜66%) of the volume has been damaged, the effect is irreversible. In that case, the parameter Ω=1 (see below/right side horizontal line H2 in FIGS. 4 to 10).

A second criterion is also introduced, to take into account the fact that when only 10% of the volume is damaged, this is not sufficient to bring about an irreversible effect, but a heating up will be generated, which can be felt by the patient (see below/right side of horizontal line H1 in FIGS. 4 to 10).

The parameters Eₐ (activation energy) and A (thermal susceptibility) are fixed by the values in table III below.

TABLE III

| Medium | Eₐ (J · mol⁻¹) | A(s⁻¹) |
|---|---|---|
| Blood (S) | 4.48 · 10⁵ | 7.6 · 10⁶⁶ |
| Wall (P) | 4.30 · 10⁵ | 5.6 · 10⁶⁶ |
| Tissue (T) | 4.30 · 10⁵ | 5.6 · 10⁶⁶ |

For the calculations, the fact is also taken into account that a laser shot into the vein takes place every 5 seconds (the interval between two shots) and that between every shot and the next, the laser beam is moved 3 mm (the distance of longitudinal withdrawal in Z of the laser fiber (f) between each shot and the next).

FIGS. 4 to 10 show via two vertical lines V1 and V2, the respective positions of the internal and external faces of wall P of the vein. To the left of the vertical line V1 is located the blood (S) and to the right of vertical line V2 is located the tissue (T).

FIGS. 4 to 10 show via two horizontal lines H1 and H2 the two aforementioned damage thresholds, namely, the horizontal line H1 which corresponds to 10% of damaged tissues and the horizontal line H2 which corresponds to 66% of damaged tissues.

The fact should be taken into account that in the zone between the two horizontal lines, the tissues are heated up and therefore partially denatured, although not irreversibly so. Outside the limit of 66% (horizontal line H2), the destruction of the tissues is irreversible.

Points A1 to A7 in FIG. 1 are obtained from iso-damage curves of FIGS. 4 to 10, taking from each figure the iso-damage curve, which intersects the limit of 66% of damage (horizontal line H2) to the left of the vertical line V2 (external face of the vein wall) located closest to the external face V2 of the vein wall P.

By interpolation, the equation of the curve linking points A1 to A7 of FIG. 1 is:

E=k₁e^{k}^{₂}^{.Dvein} (14)

### Where:

E is the laser beam energy in Joules;
Dᵥₑᵢₙ is the internal diameter in mm of the vein;
whilst k₁ (=6) and k2 (=0.5) correspond to a wavelength of 980 nm.

Of course, this mathematical equation obtained by linear interpolation and specifically the values of k1 and k2 quoted above, do not limit the invention. By following the same simulation step, but taking other iso-damage curves to establish FIG. 1, intersecting the damage limit of 66% (horizontal line H2) in the area of the vein wall P (between the two vertical lines V1 and V2), it is if necessary, possible to obtain a different equation, but one which nevertheless makes it possible to express in a simple manner the relationship with the energy needed by the laser beam (in the particular example described for obtaining essentially a 66% destruction of the tissues of the vein wall P) depending on the internal diameter of the vein. The same method can be used, but adopting a damage limit other than 66%, the limit of 66% being merely a preferential value, which makes it possible to obtain irreversible effects on the vein wall P.

Simulations have also been carried out, varying the value of the coefficient of absorption of blood μₐ, which indirectly modifies the wavelength of the laser beam. The results obtained for coefficients k1 and k2 of equation (14) appear in table IV below.

TABLE IV

| μₐ (blood) | k1 | k2 |
|---|---|---|
| 0.1 | 5.967 | 0.5765 |
| 0.15 | 5.8322 | 0.5741 |
| 0.2 | 7.503 | 0.4442 |
| 0.35 | 6.2736 | 6.2736 |

It can be seen from the graph in FIG. 1 that in the case of a vein with a too large internal diameter (typically larger than 5 mm for the internal diameter of the vein), the laser energy required to destroy the vein wall becomes too high; this is explained by the presence in the vein of a too large quantity of blood, which absorbs a lot of laser energy diffused into the blood, before it reaches the vein wall. In practice in the case of veins with a too large internal diameter (typically larger than 5 mm), the laser energy required to destroy the vein wall is too high and the treatment of this type of vein cannot accordingly be implemented using an acceptable amount of laser energy.

The invention is not limited to a calculation of the energy of the laser beam according to the internal diameter of a vein, but more generally covers every means of energy calculation (of the power or of the time of emission) of an endovenous laser beam, according to the cross-section of the vein (in particular the diameter, the radius or the surface area of the cross-section)

### Method of Treatment of Varicose Veins or the Like, Setting the Laser Beam According to the Cross-Section of the Vein

It is possible to use any known type of endovenous laser which functions in particular (but not only) using wavelengths of 800-1000 nm, regardless of the structure of the laser, or of the associated means (catheter or other) used to cause the laser beam to penetrate into the interior of the vein. The laser can be of the pulsating, or of the continuous type.

Generally speaking, the endovenous laser comprises a laser source, which makes possible the manual setting of the time of emission (tₗₐₛₑᵣ) of the beam and/or the power of the laser beam emission (Pₗₐₛₑᵣ) and of a laser fiber output by the practitioner.

The laser is used in two stages, namely:
the stage of setting the laser according to the vein to be treated
the stage of using the set laser for the endovenous treatment of the vein.

### Setting of the Laser

For the preliminary setting of the laser, the user responsible for the setting operations is supplied with a support (paper or other), comprising at least an abacus of the type shown in FIG. 1 (in the event specific to an interval of 5 seconds between shots and to a withdrawal distance of 3 mm).

The method of setting is implemented in the following stages, namely:
(a) the region of the vein to be treated (varicose vein) is located and the internal diameter of the vein in that region is measured by e.g. echography;
(b) using the abacus (FIG. 1), the energy E required for the laser beam is determined.
(c) the time of emission (tₗₐₛₑᵣ) and/or the power (Pₗₐₛₑᵣ) of the laser are set to obtain the energy (E) needed, it being recalled that:

E(J)=Pₗₐₛₑᵣ(W)·tₗₐₛₑᵣ(s)

It should be noted that the stages of setting (a) to (c) are not necessarily implemented by the phlebologist, but possibly by a technician not possessing any surgical skills.

### Treatment

When the laser has been set, the treatment of the vein takes place in the following stages, namely:
(d) using a catheter, the optical fiber of the laser is introduced into the vein up to the initial point of emission, which is the most distant from the venous region to be treated, the patient having previously undergone local or general anaesthesia; the optical fiber output is not in contact with the internal wall of the vein and the initial point of emission is preferably located at a distance of at least 1 cm and preferably at least 2 cm from the sapheno-femoral junction.
(e) a first shot is triggered.
(f) the laser optical fiber is withdrawn at a rate set by the abacus (3 mm in the case of FIG. 1) and after a lapse of a determined time interval (5 seconds in the case of the abacus in FIG. 1) a fresh shot is fired. This stage is repeated as often as is necessary in order to cover the entire length of the venous region, which is to be treated.

In this way the local and irreversible destruction of the venous wall ensues and this, in turn, leads to a preferably (but not necessarily) complete sealing off of the vein in the treated region.

In a variant of embodiment, the stage (d) is performed before the first stage (a) of setting, so that measuring the internal diameter of the vein is favourably performed when the laser fiber and its catheter are already set up in the vein.

When the blood pressure in the region to be treated deforms the vein to an excessive extent, (internal diameter of the vein too large), it is possible prior to the treatment, to incline the patient from the horizontal into the so-called Tredelenbourg position, so as to reduce the said blood pressure and thus also slightly to reduce the initial internal diameter of the vein prior to treatment.

The abacus in FIG. 1 may be replaced by an abacus, which directly furnishes the value of the settable parameter(s) of the laser source, namely, the value of the power of the laser beam (Pₗₐₛₑᵣ) for a predetermined time of emission (tₗₐₛₑᵣ), or the value of the time of emission of the laser beam (tₗₐₛₑᵣ) for a predetermined power (Pₗₐₛₑᵣ) of the laser beam, which avoids the need to calculate these parameters from the energy (E).

In another variant of the embodiment of the invention, the equation linking the energy (E) and the internal diameter of the vein may be implemented in the form of a computer programme, which is for example supplied to the user recorded on a memory support, preferably on a CD-ROM or a diskette after which the user only has to load this programme into the memory of a normal commercial micro-computer. This programme is used at stage (b) for setting the laser. This programme may for example be designed at a first stage for prompting the user via the computer screen to enter the value of the entry parameter (Dᵥₑᵢₙ) and for allowing the user via the computer keyboard to enter the said value. Using this value entered by the user the programme calculates by means of the foregoing mathematical formula, the corresponding value of energy E or if necessary, the value of power (Pₗₐₛₑᵣ) of the laser beam for a predetermined time of emission (tₗₐₛₑᵣ), or the value of the time of emission of the laser (tₗₐₛₑᵣ) for a predetermined power of the laser beam (Pₗₐₛₑᵣ).

In yet another variant of the embodiment, it is possible to design an endovenous laser, which incorporates a computer programme according to the invention, for the automatic calculation of the value of energy of the laser beam (E) or the value of the power (Pₗₐₛₑᵣ) of the laser beam for a predetermined time of emission (tₗₐₛₑᵣ), or of the value of the time of emission (tₗₐₛₑᵣ) of the laser beam for a predetermined power (Pₗₐₛₑᵣ) of the laser beam. This programme may for example be stored in an electronic memory (of the RAM or EPROM type) of the endovenous laser, the said memory being accessible for reading by a processor (for example a microprocessor or a microcontroller) of the endovenous laser.

In a simple embodiment, the setting of the parameter of functioning of the laser (energy, power or time of emission) is carried out manually from the value supplied automatically, for example on an endovenous laser screen, from the value of the diameter (Dᵥₑᵢₙ) entered into a means of calculation of the endovenous laser.

In a more sophisticated embodiment (FIG. 11), the endovenous laser comprises means 1 of automatic setting of the parameter of functioning of the laser (for example, the time of emission or power) which means of setting automatically pilot the source of laser 2 and according to a value of entry instruction (t or P) which is furnished by the means of automatic calculation 3.

1-9. (canceled)

10. A method of setting an endovenous laser for the treatment of a vein, comprising the following steps:
determining the internal diameter (or radius) of the vein to be treated,
calculating a setting value as a function of an exponential law, which depends on the internal diameter (or radius) of the vein to be treated,
using the calculated setting value for setting the energy (E) of the laser beam, or the value of the power (Pₗₐₛₑᵣ) of the laser beam for a predetermined time of emission, or the value of the time of emission (tₗₐₛₑᵣ) of the laser beam for a predetermined power of the laser beam.

11. The method of claim 10, wherein the setting value (E) is used for setting the energy (E) of the laser beam, and is calculated by means of the following equation: E=k1.e^{k2.Dvein}, where Dᵥₑᵢₙ is the internal diameter of the vein, k1 and k2 are predetermined constants.

12. The method of claim 11, wherein k1 lies between 5 and 8.

13. The method of claim 11, wherein k2 lies between 0.4 and 0.6.

14. The method of claim 10, wherein the setting value (Pₗₐₛₑᵣ) is used for setting the power of the laser beam and is calculated by means of the following equation: Pₗₐₛₑᵣ=(k1/tₗₐₛₑᵣ)e^{k2.Dvein}, where tₗₐₛₑᵣ is the time of laser emission, Dᵥₑᵢₙ is the internal diameter of the vein, k1 and k2 are predetermined constants.

15. The method of claim 14, wherein k1 lies between 5 and 8.

16. The method of claim 14, wherein k2 lies between 0.4 and 0.6.

17. The method of claim 10, wherein the setting value (tₗₐₛₑᵣ) is the time of emission of the laser beam, and is calculated by means of the following equation: tₗₐₛₑᵣ=(k1/Pₗₐₛₑᵣ)e^{k2.Dvein}, Dᵥₑᵢₙ is the internal diameter of the vein, k1 and k2 are predetermined constants.

18. The method of claim 17, wherein k1 lies between 5 and 8.

19. The method of claim 17, wherein k2 lies between 0.4 and 0.6.

20. A method of treatment of a blood-containing vein using an endovenous laser that comprises an optical fiber for delivering a laser beam, said method comprising the following steps:
a. determining the internal diameter (or radius) of the vein to be treated,
b. calculating a setting value as a function of an exponential law, which depends on the internal diameter (or radius) of the vein to be treated,
c. using the calculated setting value for setting the energy (E) of the laser beam, or the value of the power (Pₗₐₛₑᵣ) of the laser beam for a predetermined time of emission, or the value of the time of emission (tₗₐₛₑᵣ) of the laser beam for a predetermined power of the laser beam,
d. introducing the optical fiber into the interior of the blood-containing vein up to the site to be treated,
e. emitting a laser beam into the blood-containing vein.

21. The method of claim 20, wherein the setting value (E) is used for setting the energy of the laser beam, and is calculated by means of the following equation: E=k1.e^{k2.Dvein}, where Dᵥₑᵢₙ is the internal diameter of the vein, k1 and k2 are predetermined constants.

22. The method of claim 21, wherein k1 lies between 5 and 8.

23. The method of claim 21, wherein k2 lies between 0.4 and 0.6.

24. The method of claim 20, wherein the setting value (Pₗₐₛₑᵣ) is used for setting the power of the laser beam and is calculated by means of the following equation: Pₗₐₛₑᵣ=(k1/tₗₐₛₑᵣ)e^{k2.Dvein}, where tₗₐₛₑᵣ is the time of laser emission, Dᵥₑᵢₙ is the internal diameter of the vein, k1 and k2 are predetermined constants.

25. The method of claim 24, wherein k1 lies between 5 and 8.

26. The method of claim 24, wherein k2 lies between 0.4 and 0.6.

27. The method of claim 20, wherein the setting value (tₗₐₛₑᵣ) is the time of emission of the laser beam, and is calculated by means of the following equation: tₗₐₛₑᵣ=(k1/Pₗₐₛₑᵣ)e^{k2.Dvein}, Dᵥₑᵢₙ is the internal diameter of the vein, k1 and k2 are predetermined constants.

28. The method of claim 27, wherein k1 lies between 5 and 8.

29. The method of claim 27, wherein k2 lies between 0.4 and 0.6.
